(19) United States
(12) Patent Application Publication
(10) Pub. No.: US 2024/0293631 A1
(43) Pub. Date: Sep. 5, 2024

(54) CLOSED INTUBATION SYSTEMS HAVING IMPROVED AIRWAY ACCESS FOR EXAMINATION DEVICES

(71) Applicant: UNIVERSITÄT ZU KÖLN, Köln (DE)

(72) Inventors: Philipp KÖHLER, Köln (DE); Oliver Andreas CORNELY, Köln (DE); Matthias KOCHANEK, Köln (DE)

(21) Appl. No.: 18/562,644

(22) PCT Filed: May 11, 2022
(86) PCT No.: PCT/EP2022/062758

§ 371 Date: Nov. 20, 2023

(30) Foreign Application Priority Data
May 19, 2021 (DE) 10 2021 113 007.7

(51) Int. Cl. A61M 16/04; A61M 25/00; A61M 39/10

(52) U.S. Cl. CPC A61M 16/0463; A61M 25/0097; A61M 39/10; A61M 2039/1077; A61M 2205/14

(57) ABSTRACT

A closed intubation system includes at least one endotrache-al tube suitable for insertion into the trachea of a patient, a tubular connection to a ventilation device and an adapter arrangement with at least three cylindrically shaped receptacles for producing a gas-tight coupling between the endotracheal tube, the connection of the ventilation device and at least one further receptacle. The adapter arrangement includes an adaptor base body, an adaptor attachment, an eleastic tubular sheath, and an attachment connection.

### INTRODUCTION

The present disclosure relates to a closed intubation system comprising at least one endotracheal tube suitable for insertion into the trachea of a patient, a tubular connection to a ventilation device and an adapter arrangement with at least three cylindrically shaped receptacles for pro-ducing a gas-tight coupling between the endotracheal tube, the connection of the ventilation device and at least one further receptacle.

Nowadays, employees working in the healthcare system are exposed to an increasing number of personal stresses. This applies to a large extent to staff working in hospitals, particularly nursing staff with direct patient contact and intensive care staff. For the latter groups, the constant increase in workload and the general shortage of nursing staff in recent years has led to a particular increase in their own risk potential, which has been further exacerbated by the pandemic situation triggered by SARS-CoV-2, with a significant increase in intensive care case numbers and a significant increase in patient infectivity. Many artificially ventilated COVID-19 patients suffer from acute respiratory distress syndrome (ARDS), the treatment of which requires positive pressure-controlled ventilation with positive end-expiratory pressure (PEEP) and the use of humidified, temperature-controlled ventilation air. In intensive care treatment, however, life-threatening complications such as severe pulmonary bleeding (>200 ml/24 h), foreign bodies in the lungs, atelectasis or similar may require immediate bronchoscopy or bronchoalveolar lavage (BAL) for treatment and sampling. The emergency measures usually result in the closed ventilation circuit having to be opened, releasing aerosols and droplets into the ambient air, which is associated with a considerable risk of exposure to infectious diseases (SARS-CoV-2, MERS, influenza, tuberculosis, etc.) for the medical personnel directly involved. Due to the risk described above, samples are alternatively obtained from the upper respiratory tract of COVID-19 patients, which can lead to secondary infections not being diagnosed at all or being diagnosed too late. In this respect, technical aids that enable safe and simple treatment or sampling of the lungs of even highly infectious patients without risk to the treating staff would be desirable.

The patent literature also contains a wide variety of approaches to designing closed intubation systems and bronchoscopes and suction catheters that are compatible with them.

For example, U.S. Pat. No. 4,850,350 describes a closed system with a combined medical-surgical suction and ventilation tubing device comprising (1) a suction catheter, (2) a suction control valve positioned at the distal end of the system, (3) a multiple port coupling assembly through which the catheter is passed for aspiration of a patient, (4) a flexible tubular sheath providing a sterility protective sheath for the catheter attached at its distal end to the proximal end of the cou-pling unit, and (5) a sheath release valve. The coupling units have two separate elements, one of which is axially fitted into the other so that they can rotate relative to each other about their longitudinal axes to close and open access of the catheter from the sheath through the cou-pling unit into the patient. The suction control valves have first and second substantially iden-tical portions positioned along a longitudinal side in sliding engagement with each other to move between an open and a closed position of the valves. The sheath relief valves prevent air in the sheath from becoming contaminated during use of the assembly.

US 2010/0154799 A1 describes an airway access assembly comprising: a movable connector having at least one opening, the opening being in communication with an artificial airway of a patient; a closed suction catheter assembly comprising at least one connector end having an opening formed therethrough, a suction catheter, and a sheath positioned over the suction catheter; a shuttle movable between the opening in the connector and the opening in the connector end of the closed suction catheter assembly, the shuttle having an opening formed therethrough and being configured to move to a locked position and an unlocked position relative to the connector; an actuator for moving the shuttle to the locked position and the unlocked position; and a flap positioned adjacent the shuttle and the closed suction catheter assembly, the flap being movable to an open position, wherein passage of the suction catheter into an artificial airway is allowed when the shuttle is in a locked position, and the flap is movable to a closed position, wherein passage of the suction catheter through the shuttle is prevented when the shuttle is in an unlocked position.

The possibilities and importance of protecting intensive care physicians from unwanted infections caused by the escape of infectious particles during ventilation of COVID-19 patients was shown, for example, by Koehler P., Cornely O. A., Kochanek M., “Bronchoscopy safety precautions for diagnosing COVID-19 associated pulmonary aspergillosis-a simulation study” in Mycoses, 2020.

Such solutions known from the state of the art may offer further potential for improvement, particularly with regard to their suitability for reducing the exposure of ambient air in intensive care interventions under closed ventilation circuits.

### SUMMARY

It is therefore a task per an embodiment of the disclosure to at least partially overcome the disadvantages known from the prior art. In particular, it is a task per an embodiment of the disclosure to provide a closed intubation system which enables safe ventilation of a patient and simple performance of intensive medical examinations without unintentional release of aerosol particles or droplets into the ambient air.

The problem is solved per embodiments by the features of the independent claim directed to the device according to the disclosure. Preferred embodiments are indicated in the dependent claims, in the description or in the figures, whereby further features described or shown in the dependent claims or in the description or in the figures may individually or in any combination constitute an object of the disclosure, as long as the context does not clearly indicate the contrary.

According to an embodiment, a closed intubation system is thus at least comprising an endotracheal tube suitable for insertion into the trachea of a patient, a tubular connection to a ventilation device and an adapter arrangement with at least three cylindrically shaped receptacles for producing a gas-tight coupling between the endotracheal tube, the connection of the ventilation device and at least one further receptacle, wherein the adapter arrangement comprises at least:
a) an adapter base body designed in a T-shape via the arrangement of the receptacles with at least one shut-off device, the shut-off device being designed to open or close the air path between the further receptacle and the two receptacles for the ventilation device and the endotracheal tube in a gas-tight manner,
b) an adapter attachment which can be coupled to the further receptacle, wherein the adapter attachment can be connected in a gas-tight manner via a seal to the adapter base body and can be mechanically fixed to the latter via retaining means;
c) an elastic, tubular sheath arranged gas-tight at one end on the adapter attachment;
d) an attachment connected in a gas-tight manner to the other end of the elastic, tubular sheath, the attachment comprising a passage to the tubular sheath with a sealing device arranged therein, wherein the sealing device is designed to receive cylindrically shaped devices and to guide these through the attachment, the tubular sheath, the adapter attachment, the adapter base body and the endotracheal tube in a displaceable and gas-tight manner.

Surprisingly, it has been shown that the closed intubation system with the above-mentioned adapter structure can be used to ensure simple, fast and safe treatment and sampling of the airways of even highly infectious patients without droplets or aerosols escaping from the closed circuit into the ambient air. Potentially released particles are safely enclosed in the adapter arrangement or in the tubular sheath and the insertion and handling of an examination device, for example a bronchoscope, to the examination site in the patient's lungs is only restricted to a negligible extent. In this way, exposure of the environment and the people being treated to potentially infectious particles is reliably prevented. In addition to contamination safety, the adapter arrangement also ensures rapid operability, which may be particularly important in emergency medical, invasive measures. In addition, the structure according to an embodiment enables sterile handling of bronchoscopes or other examination instruments, contrary to the properties of structures known from the prior art. The adapter arrangement is also so flexible that a range of different procedures, such as bronchoscopy or lung lavage, can be performed. The special design of the arrangement also ensures that samples can be removed and isolated quickly and safely. In addition to safe treatment and occupational safety, this can also simplify the further course of the examination.

The system considered here is a closed intubation system. Endotracheal intubation is generally used when patients require mechanical respiratory support. Another possible application is to create a secure ventilation route in the absence of protective reflexes. Intubation prevents ob-struction of the upper airway and provides protection against aspiration. During intubation, a cuff (block cuff, small balloon at the end of the endotracheal tube) is inflated in the tube posi-tion to seal the trachea. This seal prevents stomach contents, blood or foreign bodies from entering the lungs, or only in small quantities. In endotracheal intubation, an endotracheal tube is inserted into the windpipe (trachea) through the mouth (orotracheal), nose (nasotracheal) or a tracheostoma. Intubation is the standard method of securing the airway and is used in anaesthesia, intensive care and emergency medicine, for anaesthetized or unconscious patients or generally in cases of acute respiratory disorders. The inserted tube can be a single or double lumen tube, which enables side-separated ventilation of the lungs. The system is a closed system in cases where there is no significant air exchange with the unprotected ambient air during forced ventilation apart from the ventilator through the intubation system.

As components, the intubation system comprises an endotracheal tube suitable for insertion into the trachea of a patient and a tubular connection to a ventilation device. The intubation system thus comprises at least one connection to a source of air for ventilating the patient. In addition to the necessary pressure generator, this device can also have other components, such as a humidifier and a temperature control unit for the breathing air. The ventilation device also has a suitable connection by means of which the breathing air is fed from the ventilation device to the patient. This can take place via a tube-like or tubular connection, which ensures both the supply and removal of the breathing air directly or indirectly to the endotracheal tube. Another component of the system is an endotracheal tube, which can be inserted into the patient's lungs. Endotracheal tubes of different designs can be used in combination with the other components of the system.

The intubation system also comprises an adapter arrangement with at least three cylindrically shaped receptacles for creating a gas-tight coupling between the endotracheal tube, the connection of the ventilation device and at least one further receptacle. The adapter arrangement, in which the supply line from the ventilation device and the connection of the endotracheal tube are brought together, serves as a mechanical and gas-tight connecting or coupling piece. Due to the tubular design of the endotracheal tube and the usual connection of the ventilation device as a tube connection, the adapter arrangement therefore has cylindrical receptacles to which the two tube ends can be fixed in a gas-tight manner. The mechanical connection of the tubes to the receptacles can be made using connection types known to the skilled person, for example by mechanical plug-in or screw connections. However, it is also possible for the receptacles to be designed independently of each other as coupling pieces, which enable a simple plug-in connection between the adapter arrangement and hoses using one or more sealants. In addition to the receptacles in the direction of the ventilation device and in the direction of the endotracheal tube, the adapter arrangement also has at least one further connection option, which is mechanically independent of the other two receptacles. It would therefore not be in accordance with the disclosure if there were only two attachment pieces or receptacles, with the air path between one or more of the ventilator, endotracheal tube and further connection components being at least partially combined before entering the adapter arrangement.

The adapter arrangement comprises at least a) an adapter base body designed in a T-shape via the arrangement of the receptacles with at least one shut-off device, the shut-off device is designed to open or close the air path between the further receptacle and the two receptacles for the ventilation device and the endotracheal tube in a gas-tight manner. The air path via the additional receptacle can be decoupled from the receptacles or connections of the endotracheal tube and the ventilation device so that there is no air flow between the additional receptacle and the two other receptacles. The shut-off device does not influence or obstruct the air flow from the ventilation device to the endotracheal tube. The shut-off device can take the form of a mechanically or electrically operated valve or closure. However, it can also be one or more reversibly closable and openable diaphragms, provided that the shut-off device can withstand a pressure difference of, for example, more than 100 Pa, more than 500 Pa and preferably more than 2.5 kPa between the different ventilation phases of forced ventilation and further admission.

Furthermore, the adapter arrangement b) comprises an adapter attachment which can be coupled to the further receptacle, wherein the adapter attachment can be connected in a gas-tight manner via a seal to the adapter base body and can be mechanically fixed to the latter via re-taining means. An adapter attachment can therefore be attached to the further receptacle of the adapter arrangement in a gas-tight manner, which can be mechanically coupled to the further receptacle of the adapter arrangement via a plug-in connection, for example. The gas-tight connection can also be made via a coupling connection. The connectivity of the adapter attachment ensures that the other parts of the adapter arrangement are only connected to the system when they are actually needed. If pure ventilation of the patient without further examinations is desired, the further adapter can be hygienically sealed off from the environment by means of a simple mechanical seal. The adapter attachment can, for example, have a cylindri-cal or conical symmetry and an extension along its axis of symmetry of, for example, 1 cm to 10 cm.

In addition, the adapter arrangement c) comprises an elastic, tubular sheath arranged gas-tight with one end on the adapter attachment. The adapter attachment can be connected to the adapter arrangement at one end and is connected to a sheath at its other end. The sheath can be connected to the adapter attachment from the outside, for example. The cover can, for example, encompass the entire adapter attachment and be glued or clamped to it from the outside. However, it is also possible for the tubular cover to be attached or fastened to the inner edge of the adapter attachment. It is important that the tubular cover is connected to the adapter attachment in a gas-tight manner, per embodiments. A tubular sleeve is a mechanically flexible and movable attachment, which can be realized, for example, by a thin plastic tube or sleeve. The sleeve is elastic in cases where the sleeve can be reversibly compressed and stretched again. For example, an elastic sleeve is created in cases where the sleeve can be compressed to 1/10 of its original length and stretched out again several times. Preferably, the elastic sleeve can be made of PE or PVC or comprise these polymers. The elastic sheath can preferably have a diameter of greater than or equal to 0.5 cm and less than or equal to 5 cm, furthermore preferably greater than or equal to 1.0 cm and less than or equal to 2.5 cm. The elastic sheath can preferably have a longitudinal extension of greater than or equal to 15 cm and less than or equal to 100 cm, furthermore preferably greater than or equal to 50 cm and less than or equal to 90 cm. The elastic sleeve can, for example, be attached to the adapter attachment in a compressed form for storage when not in use.

Finally, the adapter arrangement d) comprises an attachment connected in a gas-tight manner to the other end of the elastic, tubular sheath, wherein the attachment comprises a passage to the tubular sheath with a sealing device arranged therein, wherein the sealing device is designed to receive cylindrically shaped devices and to guide these through the attachment, the tubular sheath, the adapter attachment, the adapter base body and the endotracheal tube in a displaceable and gas-tight manner. A further attachment is coupled to the other end of the tubular sheath, which can be connected to the tubular sheath by means of an adhesive or a clamp, for example. This attachment has a passageway to the inside of the tubular sleeve, with at least one further sealant being fitted in the passageway. The sealing agent of this sealing device can be an O-ring, for example. However, it is also possible for the seal to be realized via a flap arrangement. The flap arrangement can open when a mechanical force is applied, for example by inserting a bronchoscope, and allow the bronchoscope to pass through the sealing device of the attachment into the interior of the sheath. The bronchoscope can thus be pushed through the sealing device in the direction of the examination site, gas-tight against the environment. The sealing device as the final seal against the environment prevents potentially in-fectious particles or aerosols from escaping unintentionally. The infectious particles or aerosols are retained in the tube-like sheath at the latest. The attachment can also have means for reversibly fixing the attachment to the adapter attachment or to the adapter arrangement. For example, the attachment can be held on these components by a magnetic interaction. As a result, the attachment can be coupled to the sheath if required and the examination and/or treatment can be started by inserting the examination devices through the seal of the attachment and through the tubular sheath. The attachment can, for example, be cylindrical in shape, whereby the internal diameter of the cylinder can, for example, be greater than or equal to 0.5 cm and less than or equal to 5 cm, and furthermore preferably greater than or equal to 1 cm and less than or equal to 3.0 cm.

In an embodiment of the intubation system, the shut-off device can comprise a rotatable closure mechanism consisting of a substantially cylindrically designed base body with a substantially centrally arranged passage, wherein at least two sealing means spaced apart along the axis of symmetry can be arranged in the passage. For secure mechanical guidance and improved handling of a bronchoscope or a suction device, the use of two sealing means within the opening of the cylindrically shaped base body of the shut-off device has proven to be particularly suitable. Within this critical section of the adapter arrangement, the inserted examination object is held mechanically in a particularly secure manner. In addition, the double seal design contributes to an improved seal against the other adapter arrangement remote from the patient. The use of a double seal at this point is not obvious, as the skilled person would have to assume that a double seal would prevent the bronchoscope from moving freely and thus impair critical handling properties too much. It could also be assumed that the removal of biological samples from the lungs would be made more difficult, as the passage through the seals would now have two constrictions. Surprisingly, neither is the case and so the improved sealing effect remains between the closed breathing air circuit and the receptacle for the examination device. The two seals can be designed in the form of O-rings, for example.

Within a further embodiment of the intubation system, the cylindrically designed base body of the rotatable closure mechanism can comprise a conical recess in the centrally arranged passage in the direction of the further receptacle, whereby the cone angle can be greater than or equal to 20° and less than or equal to 90°. Attaching a cone-shaped recess to the locking mechanism can significantly increase the handling properties, for example of bronchoscopes for examining the lungs. On the one hand, inserting and passing the bronchoscope through the opening can be made much easier. On the other hand, the cone can increase the lateral mobility of the bronchoscope, which contributes to better manoeuvrability of cylindrical suction devices or bronchoscopes. These improved properties result in particular in conjunction with a double seal within the opening, whereby the double seal can also contribute in particular to improved sealing even with higher pressure differences or rapid pressure changes. In a further embodiment, the cone angle can be greater than or equal to 30° and less than or equal to 85°, furthermore preferably greater than or equal to 45° and less than or equal to 75°.

Within a further aspect of the intubation system, the shut-off device in the adapter base body may comprise at least one locking means, wherein the locking means may be arranged to hold the shut-off device in a closed position. For the safety of the closed ventilation circuit, it has proven to be advantageous that the shut-off device of the adapter arrangement is initially held in a closed position. In this position, no air exchange between the other adapter and the components of the ventilation circuit is possible. In this position, an examination instrument cannot be inserted into the endotracheal tube. The locking means can be, for example, a spring or other mechanical locking means that prevents accidental opening of the locking device without significant and deliberate force. The spring force can only be overcome and access for inserting the examination instrument into the endotracheal tube released after a minimum amount of force has been applied. In the open position, a bronchoscope, for example, can then be inserted into the endotracheal tube. This design can also prevent accidental opening of the ventilation circuit in hectic situations. In addition to the spring, a spring lock can also be fitted, which can, for example, mechanically hold the spring in a corresponding groove. Advantageously, the design of the shut-off device can actively indicate whether the opening is closed or open. The shut-off device can also be made of a transparent material which, in addition to the visual check of the open/closed position, also allows the operation to be checked for possible blockages.

According to a characteristic of the intubation system, the shut-off device in the adapter base body can comprise at least one detection means for detecting an object inside the passage of the shut-off device. In order to increase the safety of use, it has proved to be particularly advantageous for the shut-off device in the adapter base body to have a detector which can detect an object in the passage of the shut-off device. The detection means can be a simple mechanical switch, for example, which triggers on mechanical contact, for example with a bronchoscope. However, the detection means can also be of an electrical or optical type, in which case the electrical or optical signal also indicates the presence of an object in the passage of the shut-off device. If the detection means is triggered, the closing of the shut-off device can be blocked, for example. In addition, the detection means can indicate the earliest possible time at which the shut-off device can be safely closed. This can prevent unintentional jamming of biological material in the opening of the shut-off device. The detection means can also ensure that the shut-off device closes safely in any case.

In a further embodiment of the intubation system, the connection of the elastic sheath to the adapter attachment as well as the connection of the elastic sheath to the attach-ment can each be designed to be individually rotatable. For particularly easy handling of a suction device or a bronchoscope, it can be helpful that the points of the intubation system where a tight seal to the inserted object is important are designed to be rotatable. This can facilitate the handling of a suction catheter, for example, as there is no fixed relationship between the intubation system and the catheter at these points. This results in a greater degree of mechanical freedom and, in particular, prevents unintentional twisting of the elastic sheath. Furthermore, the mechanical forces acting on the adapter arrangement are reduced. By reducing the mechanical forces, the surgeon can also manipulate the suction catheter or bronchoscope more precisely and easily.

Within an aspect of the intubation system, at least a part of the adapter base body of the adapter base body can be made of a transparent material. For better control of handling and for simple initial assessment of removed biological material, it has been found to be particularly suitable for the adapter base body to consist at least in part of an optically transparent material. Suitable materials can, for example, be selected from the group of plastics, whereby the skilled person is familiar with sufficiently mechanically strong materials from this group. In this embodiment, either the entire adapter base body or only a partial area, for example one half, can consist of or have the transparent material.

In an embodiment of the intubation system, the adapter base body comprises means for detecting the applied pressure and means for controlling the shut-off device, whereby the opening or closing of the shut-off device can be controlled as a function of the applied pressure. In order to optimize the opening time of the shut-off device, it has proved advantageous for the adapter base body to be equipped to detect the pressure currently applied to the ventilation device and to enable the shut-off device to be controlled as a function of the applied pressure. For example, it is conceivable that the shut-off device cannot be opened if pressure peaks are currently present. For example, the shut-off device can only be released when a low pressure is currently present during the ventilation cycle. This can reduce the pressure loss when the shut-off device is opened and, in particular, also help to ensure that the smallest possible amount of aerosols or droplets can escape through the opening of the shut-off device when the shut-off device is opened. The suitable means can be, for example, an electronic pressure sensor with integrated or external electronic control of the shut-off device. In principle, however, it is also possible for the shut-off device to be locked via a mechanical switch which moves as a function of the pressure cycle currently present in the adapter arrangement and only allows the shut-off device to be released at lower pressures. In advantageous cases, the adapter arrangement can also be electrically coupled to the ventilation device. In addition to this basic control, further mechanical or electrical means can be provided which can override this basic pressure-dependent control in emergencies. In these cases, the operator can intervene immediately in an emergency regardless of the current stage of the ventilation cycle.

In a further embodiment of the intubation system, a sealing cap with a sealing device can be arranged on the adapter base body via a mechanical connecting means, whereby the sealing cap can be designed to mechanically close the further receptacle of the adapter base body when the adapter attachment is not coupled. In everyday clinical practice, it has proven to be advantageous that the additional receptacle is in principle only arranged on the adapter base body via an additional sealing cap, which does not allow the insertion of an examination instrument, with a corresponding sealing device. In these cases, the actual adapter attachment with insertion option is only fitted directly before the upcoming examination and opening of the shut-off device. The basic adapter body is hygienically protected by the sealing cap with sealing device. A suitable mechanical connecting means for the connection between the sealing cap and the adapter base body is, for example, a cord or a polymer strip, which connects the sealing cap and adapter base body to each other in a movable manner. After the end of the examination, for example after removing the suction device, the sealing cap with sealing device can be placed back on the adapter base body.

In a further embodiment of the intubation system, the elastic, tubular sheath can comprise means for isolating a biological sample. As part of a patient examination using a bronchoscope or also as part of a suction procedure, it may be part of the clinical routine that biological samples are taken from the lung cavity. At present, these samples still have to be handled in a cumbersome manner, with the biological samples usually being exposed to ambient air for a short time. In order to eliminate this source of contamination, it has proven to be advantageous that the tube-like cover can be used as a sample container at the end of the ex-amination. For this purpose, the entire tube-like envelope or only a part of the tube-like envelope can be designed to be detachable, so that after the biological sample from the lung has been introduced into the tube-like envelope, this area of the tube-like envelope can be isolated from the environment. In this respect, the tube-like sheath can have at least two, preferably three, more preferably four individual elements spaced apart from each other, which can close the tube-like sheath locally. In this embodiment, a closure can serve to prevent the adapter arrangement from being exposed to the ambient air. The second and third closure options can be used to isolate the biological sample. The fourth closure option can be used to isolate the tubular sheath in the direction of the suction catheter or bronchoscope. The entire system can therefore be protected from uncontrolled contact with the ambient air and a biological sample can also be isolated and removed separately using these four closure options for the tube-like sheath. One possibility for designing the insulation can be, for example, the arrangement of several clips which are able to seal the tube-like cover gas-tight at different points. One or more of the clips can also have a cutting device, for example, which enables the middle section with the biological sample to be cut off. This can then be passed on isolated from the ambient air.

### BRIEF DESCRIPTION OF THE FIGURES

Further advantages and advantageous embodiments of the objects according to embodiments of the disclosure are illustrated by the drawings and explained in the following description. It should be noted that the drawings are descriptive only and are not intended to limit the invention.

It shows the:

FIG. 1 schematically shows the structure of the closed intubation system according to an embodiment;

FIG. 2 schematically shows the structure of the adapter arrangement according to an embodiment;

FIG. 3 schematically shows the structure of the basic adapter body according to an embodiment with connectable adapter attachment;

FIG. 4 schematically shows the structure of the adapter base body according to an embodiment;

FIG. 5 schematically shows the structure of the adapter attachment that can be coupled according to an embodiment with an elastic, tubular cover and a gas-tightly connected attachment.

### DETAILED DESCRIPTION

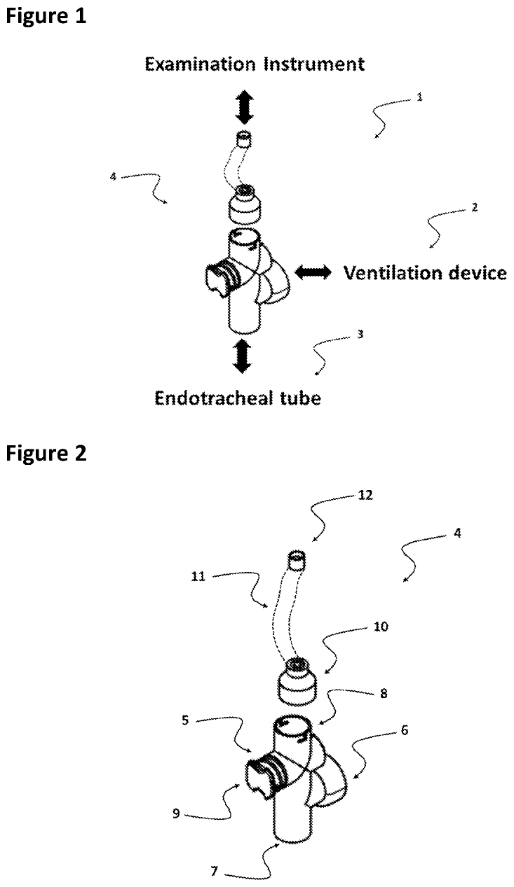

FIG. 1 shows the structure of the closed intubation system 1 according to an embodiment. The closed intubation system 1 comprises a connection to a ventilation device 2, an endotracheal tube 3 and the adapter arrangement 4 according to an embodiment. The ventilation device 2 is set up to supply a patient with possibly tempered and humidified breathing air and, in particular, oxygen. For this purpose, air or a specifically composed air mixture is fed into the patient through the closed intubation system 1 via a controlled pressure cycle. The ventilating device 2 is connected to the adapter arrangement 4 according to an embodiment by means of one or more hoses, with the hose of the ventilating device 2 being connected to a receptacle of the adapter arrangement 4. An endotracheal tube 3 can be connected to the second receptacle of the adapter arrangement 4, whereby the tube can be inserted into the patient's lungs to ventilate the patient. The adapter arrangement 4 also has a further receptacle to which an examination instrument can be connected via the adapter arrangement 4. The examination instrument can, for example, be a bronchoscope or a suction device, whereby the examination instruments are designed to be inserted at least partially into the patient's lungs. The examination instrument is inserted into the patient's lungs through the endotracheal tube 3 via the adapter arrangement 4. In particular, the design of the adapter attachment 10 of the adapter arrangement 4 allows the examination instrument to be guided flexibly and safely, whereby in particular no potentially contaminated aerosols, particles or droplets can enter the environment during the examination. This closed intubation system 1 provides particularly efficient protection for medical personnel against infection in the event of an examination. The closed intubation system 1 can be used flexibly and can be handled safely even under hectic examination condi-tions.

FIG. 2 schematically shows the structure of the adapter arrangement 4 of the closed intubation system 1 according to an embodiment. The adapter arrangement 4 comprises a basic adapter body 5, which is T-shaped via the three receptacles to the tube 3, to the ventilation device 2 and to the examination instrument. Accordingly, the adapter arrangement 4 has a receptacle 6 for connection to the ventilation device 2 and a receptacle 7 for connection to the endotracheal tube 3. These two receptacles 6, 7 can be closed gas-tightly against the other receptacle 8 via a shut-off device 9. An adapter attachment 10 can be arranged on the adapter base body 5 on the further receptacle 8 in the event of an examination to be carried out. However, this receptacle 8 can be closed with a cap with a sealing device in cases where no examination is being carried out. The adapter attachment 10 can therefore only be placed on the adapter base 5 and mechanically fixed just before an examination is carried out. In this figure, tongue and groove means are shown for this purpose, which can be used for mechanical fixation between the adapter base body 5 and the adapter attachment 10. The adapter attachment 10 can also be connected to the adapter base 5 in a gas-tight manner via an internal seal. A tube-like sheath 11 is arranged on the adapter attachment 10 and connected to it in a gas-tight manner. The tube-like sheath 11 is used to flexibly hold the front part of the examination instrument and can also hold biological samples from the patient. When the examination instrument is inserted, the tube-like sheath 11 prevents aerosols or particles from being released from the closed intubation system 1 into the environment. Although the aerosols can escape into the area of the adapter base body 5 and possibly through the adapter attachment 10, they are safely enclosed by the tube-like sheath 11 and the attachment 12 attached to it in conjunction with the examination instrument. Such isolation from aerosols or particles cannot be achieved if only the examination instrument as such is encapsulated in a displaceable casing. It is important per embodiments to isolate aerosols in an area in which the examination instrument still isolates the environment from the closed intubation system 1. For this purpose, the attachment 12 is connected to the examination instrument in a gas-tight manner via a seal. The insertion of the front part of the examination instrument via the attachment 12, the tube-like sheath 11 and the adapter attach-ment 10 into the intubation system 1, which is closed per se, takes place via the shut-off device 9. The shut-off device 9 can be designed to be rotatable, for example, and has an opening for the examination instrument to pass through. After opening the shut-off device 9, the examination instrument can be inserted through the receptacle 7 into the endotracheal tube 3 arranged on it.

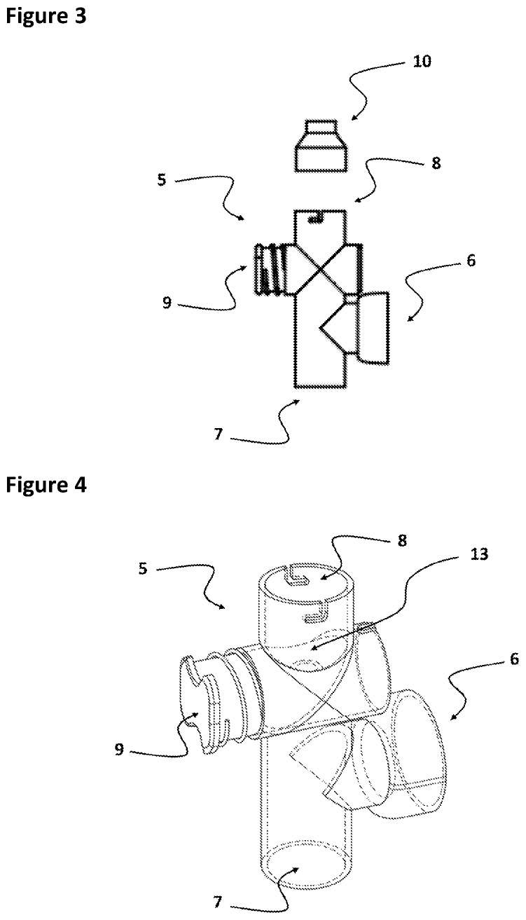

FIG. 3 schematically shows the structure of the basic adapter body 5 according to an embodiment with connectable adapter attachment 10. The basic adapter body 5 has a receptacle 6 for the ventilation device and a receptacle 7 for the endotracheal tube 3. These two receptacles can be blocked off from the receptacle 8 towards the examination instrument by the blocking device 9. The figure also shows the adapter attachment 10, which can be connected to the adapter base 5 in a gas-tight manner via an internal sealing device. The shut-off device 9 has one or more seals on the inside (not shown), through which the examination instrument can be pushed in the event of an examination to be carried out. The shut-off device 9 can also have a locking means in which the shut-off device 9 is locked in the open position (not shown). Furthermore, the shut-off device 9 can be held in the closed position, for example by a spring, whereby the spring force must first be overcome in order to open the shut-off device 9. This can prevent the shut-off device 9 from opening too easily or accidentally.

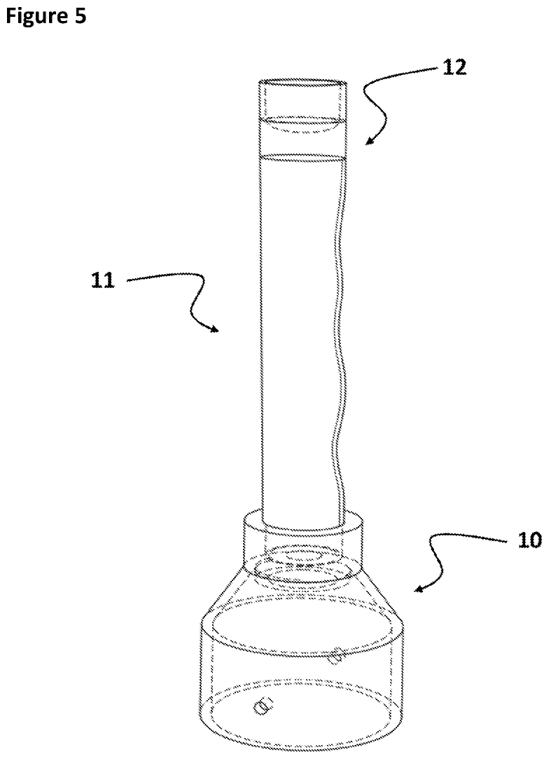

FIG. 4 schematically shows the structure of the basic adapter body according to an embodiment with the receptacle 6 for the ventilation device 2, the receptacle 7 for the endotracheal tube 3 and the further receptacle 8 for connecting the adapter attachment 10 (not shown). The basic adapter body 5 can be subdivided via the shut-off device 9. In this case, the shut-off device 9 is cylindrical and has a centrally arranged passage with two internal sealing devices, for example in the form of O-rings or flap seals. A cylindrical examination instrument can be guided or pushed through the seals in a gas-tight manner. In this figure, it can be seen that the centrally arranged passage 13 has a conical recess in the direction of the other receptacle. In this case, the cone angle is approx. 20-40°. By forming a conical recess, handling and, in particular, the insertion of the examination instrument into the tube without contact can be significantly facilitated. In addition, this design enables faster and more targeted insertion of the examination instrument, even under time pressure. Without being bound by theory, this is probably due to the fact that the lower shape of suction devices or bronchoscopes can be guided more easily to the actual opening thanks to their conical design. This can occur, for example, even when the shut-off device 9 is not fully open, which then causes an additional force to be exerted by the examination instrument to open the shut-off device 9. The opening is also covered more efficiently by the examination instrument, which reduces the spread of aerosols or droplets from the patient's lungs in the direction of further intake 8.

FIG. 5 schematically shows the structure of the adapter attachment 10, which can be coupled according to an embodiment, with an elastic, tubular sheath 11 and a gas-tightly connected attachment 12. The tubular sheath 11 can, for example, have a diameter of greater than or equal to 1.5 cm and less than or equal to 5 cm and a length of greater than or equal to 10 cm and less than or equal to 100 cm. The tubular sheath 11 is connected to both the adapter attachment 10 and the attachment 12 in a gas-tight manner by means of clamping or bonding. The tubular sheath 11 can be pushed together and pulled out so that the length of the tubular sheath 11 can be changed during the examination. The tubular sheath 11 can also have devices for dividing it into several compartments. The attachment 12 and the adapter attachment 10 each have at least one seal in their interior, which is adapted or adaptable to the diameter of the examination device. Different versions with different seal diameters can also be produced. Once the examination is complete, for example, a bronchoscope is removed from the patient's lungs by pulling the bronchoscope towards the attachment 12. The bronchoscope passes through the adapter body 5 and is then guided through the first seal and then through the second seal of the shut-off device 9. In this case, any aerosols that may be released are retained in the adapter base 5, as the bronchoscope seals against the sealant of the adapter attachment 10. If the bronchoscope is also passed through this seal, any remaining aerosols are retained in the tube-like sheath 11. The shut-off device 9 can be closed in this position or after the instrument has been passed through the seals of the adapter base 5. This reduces the risk of further release of aerosols from the closed intubation system 1. The tube-like sheath 11 can then be divided into compartments by optional constriction options after the bronchoscope itself has been partially passed through, whereby any material removed from the lungs can be used for further examination. Finally, the bronchoscope is passed through the sealing attachment 12. The adapter arrangement according to the invention can be closed on the attachment 12 by a further cap and disposed of.

As used herein, the terms “general,” “generally,” and “approximately” are intended to account for the inherent degree of variance and imprecision that is often attributed to, and often accompanies, any design and manufacturing process, including engineering tolerances, and without deviation from the relevant functionality and intended outcome, such that mathematical precision and exactitude is not implied and, in some instances, is not possible.

All the features and advantages, including structural details, spatial arrangements and method steps, which follow from the claims, the description and the drawing can be fundamental to the invention both on their own and in different combinations. It is to be understood that the foregoing is a description of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. Closed intubation system comprising at least one endotracheal tube suitable for insertion into the trachea of a patient, a tubular connection to a ventilation device and an adapter arrangement with at least three cylindrically shaped receptacles for producing a gas-tight coupling between the endotracheal tube, the connection of the ventilation device and at least one further receptacle, wherein the adapter arrangement comprises at least:
a) an adapter base body designed in a T-shape via the arrangement of the receptacles with at least one shut-off device, the shut-off device being designed to open or close the air path between the further receptacle and the two receptacles for the ventilation device and the endotracheal tube in a gas-tight manner,
b) an adapter attachment which can be coupled to the further receptacle, wherein the adapter attachment can be connected in a gas-tight manner via a seal to the adapter base body and can be mechanically fixed to the latter via retaining means;
c) an elastic, tubular sheath arranged gas-tight with one end on the adapter attachment;
d) an attachment connected in a gas-tight manner to the other end of the elastic, tubular sheath, the attachment comprising a passage to the tubular sheath with a sealing device arranged therein, wherein the sealing device is designed to receive cylindrically shaped devices and to guide these through the attachment, the tubular sheath, the adapter attachment, the adapter base body and the endotracheal tube in a displaceable and gas-tight manner.

2. Intubation system according to claim 1, wherein the shut-off device comprises a rotatable closure mechanism consisting of a substantially cylindrically designed base body with a substantially centrally arranged passage, wherein at least two sealing means spaced apart along the axis of symmetry are arranged in the passage.

3. Intubation system according to claim 2, wherein the cylindrically designed base body of the rotatable closure mechanism comprises a conical recess in the centrally arranged passage in the direction of the further receptacle, wherein the cone angle is greater than or equal to 20° and less than or equal to 90°.

4. Intubation system according to claim 1, wherein the shut-off device in the adapter base body comprises at least one locking means, wherein the locking means is arranged to hold the shut-off device in a closed position.

5. The intubation system according to claim 2, wherein the shut-off device in the adapter base body comprises at least one detection means for detecting an object inside the passage of the shut-off device.

6. Intubation system according to claim 1, wherein the connection of the elastic sheath to the adapter attachment as well as the connection of the elastic sheath to the attachment are each designed to be individually rotatable.

7. Intubation system according to claim 1, wherein at least a part of the adapter base body is made of a transparent material.

8. Intubation system according to claim 1, wherein the adapter base body comprises means for detecting the applied pressure and means for controlling the shut-off device, wherein the opening or closing of the shut-off device is controllable as a function of the applied pressure.

9. Intubation system according to claim 1, wherein a sealing cap with a sealing device is arranged on the adapter base body via a mechanical connecting means, wherein the sealing cap is designed to mechanically close the further receptacle of the adapter base body when the adapter attachment is not coupled.

10. The intubation system according to claim 1, wherein the elastic tubular sheath comprises means for isolating a biological sample.
